(19) United States
(12) Patent Application Publication
(10) Pub. No.: US 2005/0233944 A1
(43) Pub. Date: Oct. 20, 2005

(54) Arginine compositions for coordinate modification of multiple cardiovascular risk factors

(76) Inventor: John G. Babish, Brooktondale, NY (US)

Correspondence Address: McDermott Will & Emery, Suite 2200, 201 South Biscayne Boulevard, Miami, FL, 33131-4336, US

(21) Appl. No.: 11/141,085

(22) Filed: May 31, 2005

Related U.S. Application Data
(63) Continuation of application No. 10/234,002, filed on Aug. 29, 2002, now abandoned.
(60) Provisional application No. 60/316,685, filed on Aug. 31, 2001.

(51) Int. Cl. A61K 38/16; A61K 31/714; A61K 31/198; A61K 31/525; A61K 31/135

(52) U.S. Cl. 514/2; 514/724; 514/565; 514/251; 514/52

(57) ABSTRACT

The present invention relates generally to compositions and methods for coordinate reduction of the serum levels of cardiovascular arterial disease risk factors, such as total cholesterol, LDL cholesterol, HDL/LDL ratio, triglycerides, homocysteine, and C-reactive protein. The composition comprises an arginine compound and another member selected from high molecular weight aliphatic alcohol or methyl donor cofactor, such as folic acid, vitamin B6, vitamin B12 or derivatives thereof. The compositions function coordinately to modify multiple serum cardiovascular risk factors.

### RELATED APPLICATIONS

This application claims priority under 35 U.S.C. 119(e) from Provisional Application No. 60/316,685, filed on Aug. 31, 2001.

### BACKGROUND OF THE INVENTION

1. Field of the Invention

The present invention relates generally to compositions and methods for modification of multiple cardiovascular risk factors including serum levels of total cholesterol, LDL cholesterol, HDL cholesterol, triglycerides, homocysteine, and C-reactive protein.

2. Description of the Related Art

In recent years, people have tended to become less physically active and consume food that has high fat content. Such sedentary lifestyles and excessive lipid ingestion cause obesity and, along with it, a variety complications, for instance, heart and circulatory disease, respiratory disease, and diabetes. Significant risk factors for heart and circulatory disease include increases in serum levels of total and LDL cholesterol, LDL to HDL ratios, triglycerides, homocysteine and C-reactive protein.

Cardiovascular arterial disease (CAD) is the major cause of morbidity and mortality throughout the United States and the industrialized world, accounting for more deaths annually than any other disease, including cancer. The annual cost to Americans from CAD is at least $150 billion in lost wages, productivity, and treatment. A direct positive relationship between coronary artery disease and hypercholesterolemia has been documented in numerous epidemiological and clinical studies. Reduction of cholesterol and LDL cholesterol by dietary and pharmacologic intervention reduces CAD and its clinical consequences.

High serum cholesterol levels can result from either an increase in cholesterol-rich low-density lipoprotein (LDL), commonly caused by high cholesterol or saturated fat intake, or through genetic predisposition. Oxidized LDL-cholesterol promotes atherogenesis through attachment to the endothelium of the vasculature, uptake by macrophages, and transmigration into the subendothelial area creating the initial foam cell. This extraluminal accumulation of cholesterol-dense plaque can eventually reduce luminal size and result in a critical stenotic lesion that can reduce myocardial blood flow and produce ischemia. Activation of platelets and other clotting mechanisms can produce a thrombus, one that completely obstruct an artery, leading to acute myocardial infarction.

Interventional prospective clinical trials targeting lowering cholesterol have conclusively and consistently demonstrated both clinical reduction in CAD events and angiographic regression of CAD. An approximate two percent reduction in CAD events has been observed for a one percent reduction in total or LDL-cholesterol.

The role of high-density lipoprotein cholesterol (HDL-cholesterol) involves the three major interconnected pathways in lipoprotein metabolism: (1) the transport of dietary or exogenous fat; (2) the transport of hepatic or endogenous fat; and (3) reverse cholesterol transport. These pathways are interdependent and disruptions in one will affect the function and products of the others. HDL-cholesterol appears to have cardioprotective properties because of its involvement in certain processes such as reverse cholesterol transport and inhibition of LDL-cholesterol oxidation. Agents that increase HDL-cholesterol can be highly beneficial for patients at risk of CAD.

The importance of plasma triglycerides is a risk factor for the development of CAD has been controversial for many years. In univariate analysis plasma triglycerides predict CAD in several studies, but this effect is attenuated when HDL-cholesterol is included in the analysis. The controversy can be explained by the complexity of lipoprotein metabolism. Plasma triglycerides serve as markers for metabolic and clinical conditions associated with increased CAD risk. Several recent studies provide new hints of the importance of triglycerides in the development of CAD. The risk depends on their level in plasma: there appears to be an optimal concentration <1.1 μmol/L, an intermediate range with increased risk at values of 1.1 to 4 μmol/L, above the intermediate range the risk increases. Very high levels of triglycerides confer an increased risk of pancreatitis.

Conventional risk factors for CAD include the hyperlipidemia variables previously discussed, smoking, hypertension, diabetes and an adverse family history. Additional CAD risk factors discovered more recently include homocysteine and C-reactive protein. Plasma vitamins B6, B12 and folic acid are strong correlates of serum homocysteine. Normal serum homocysteine levels range from 5 to 15 μmol/L in fasting subjects. Hyperhomocysteinemia is defined as any value above the 95th percentile or more than two standard deviations above the mean values obtained from healthy, fasting control subjects. Elevated homocysteine levels are classified as moderate (15 to 30 μmol/L) intermediate (>30 pmol/L to 100 μmol/L) or severe (>100 pmol) based on fasting blood samples. Using this definition of hyperhomocysteinemia, its prevalence in the general population is five percent. Between 13 and 47 percent of people with symptomatic atherosclerotic disease, however, have been reported to have hyperhomocysteinemia.

A growing body of evidence supports the concept that local and systemic inflammation plays a role in the initiation and progression of atherosclerosis, and its complications. This fact has generated a great deal of interest in identifying markers that can be detected in the blood and could reflect the state of the underlying inflammation present in the vascular wall. C-reactive protein as a sensitive, nonspecific marker of inflammation has been widely studied. Only recently have sensitive assays been developed that allow within and between subject variation in C-reactive protein to be studied.

C-reactive protein levels are regulated primarily by the function of cytokines, such as interleukin-6 (IL-6). The function of C-reactive protein is not fully understood. It binds to a wide variety of substances, such as microbial polysaccharide, phosphatidylcholine, and damaged cell membranes. C-reactive protein also enhances the activity of phagocytic cells and activates the classical complement pathway. Of more interest with regard to atherosclerosis, the aggregated C-reactive protein can selectively bind to LDL particles in vitro, which may possibly be a factor in the pathogenesis of atherosclerosis. C-reactive protein has also been extracted from human atherosclerotic lesions.

It has been shown that C-reactive protein has a useful prognostic utility in a patient with myocardial infarction and unstable angina. Furthermore, several prospective studies have shown C-reactive protein is a predictor of increased risk for myocardial infarction, stroke or peripheral vascular disease in asymptomatic individuals with no known coronary artery disease. It has also been demonstrated that using aspirin, as an anti-inflammatory agent, reduces the risk of myocardial infection in a patient with initially elevated C-reactive protein levels.

At this time, there are no drugs or dietary supplements that can adequately address all of the CAD risk factors in a coordinate or uniform manner. Therefore, compositions and methods of use thereof to facilitate a reduction in multiple CAD risk factors are needed and will be appreciated. Such compositions or methods should preferably be capable of accomplishing these goals without requiring additional dietary restriction and function coordinately to reduce multiple CAD risk factors simultaneously.

### SUMMARY OF THE INVENTION

The preferred embodiments provide compositions that coordinately attenuate multiple CAD risk factors. The compositions comprise, as a first component, at least one arginine compound or conjugate thereof, and as a second component at least one member selected from the group consisting of high molecular weight aliphatic alcohol and methyl donor cofactor and conjugates thereof.

A certain embodiment is a composition comprising as a first component, at least one arginine compound or conjugate thereof, and as a second component, at least one compound selected from the group consisting of 1-octacosanol, 1-triacontanol, 1-dotriacontanol, folic acid, vitamin B6, vitamin B12, trimethylglycine, 5-methyl tetrahydrofolate and methylene tetrahydrofolate.

The preferred embodiments also provide methods for dietary supplementation to a warm blooded animal comprising administering to the animal an effective amount of a composition comprising, as a first component, at least one arginine compound or conjugate thereof, and as a second component at least one member selected from the group consisting of high molecular weight aliphatic alcohol and methyl donor cofactor and conjugates thereof. The methods provide for the administration to be continued until serum levels of cholesterol, triglyceride, homocysteine and C-reactive protein have decreased; and the serum level of HDL-cholesterol has increased.

The preferred embodiments contemplate therapeutic treatment of hypercholesterolemia and other CAD risk factors including hypertriglyceridemia, hyperhomocysteinemia and elevated levels of C-reactive protein.

The preferred embodiments further contemplate prevention of hypercholesterolemia and other CAD risk factors including hypertriglyceridemia, hyperhomocysteinemia and elevated levels of C-reactive protein.

### DETAILED DESCRIPTION OF THE PREFERRED EMBODIMENT

Before the present composition and methods of making and using thereof are disclosed and described, it is to be understood that this invention is not limited to the particular configurations, as process steps and materials may vary somewhat. It is also intended to be understood that the terminology employed herein is used for the purpose of describing particular embodiments only and is not intended to be limiting. It must be noted that, as used in this specification and the appended claims, the singular forms “a,” “an,” and “the” include plural referents unless the context clearly dictates otherwise.

Preferred embodiments are directed towards compositions and methods of use thereof for coordinate reduction of multiple risk factors relating to CAD. The compositions of the preferred embodiments can be formulated as dietary supplements or as therapeutic formulations. The composition comprises, as a first component, at least one arginine compound or conjugate thereof, and as a second component, at least one member selected from the group consisting of high molecular weight aliphatic alcohol and methyl donor cofactor and conjugates thereof.

The preferred embodiments provide therapeutic treatment and prevention of multiple risk factors relating to CAD. Certain CAD risk factors are hypercholesterolemia, hypertriglyceridemia, hyperhomocysteinemia, and elevated levels of C-reactive protein. The methods provide for the administration to be continued until serum levels of cholesterol, triglycende, homocysteine and C-reactive protein have decreased; and the serum level of HDL-cholesterol has increased.

As used herein, the term “dietary supplement” refers to compositions consumed to affect structural or functional changes in physiology. The term “therapeutic composition” refers to any compounds or combinations of compounds administered to treat or prevent a disease.

As used herein, the term “cardiovascular disease” refers to disease of the blood vessels of the circulatory system caused by abnormally high concentrations of lipids, homocysteine and C-reactive protein in the vessels.

As used herein, the term “hypercholesterolemia” is a condition with elevated levels of circulating total cholesterol, LDL-cholesterol, and VLDL-cholesterol as per Guidelines of the National Heart, Lung and Blood Institute at the National Institutes of Health (NIH publication No. 93-3096).

As used herein, the term “hyperlipidemia” is a condition where the serum lipid parameters are elevated. This condition manifests as an abnormally high concentration of fats. The serum lipid fraction comprises total cholesterol, low density lipoproteins, very low density lipoproteins and triglycerides.

As used herein, the term “lipoprotein” such as VLDL, LDL and HDL, refers to a group of proteins found in the serum, plasma and lymph which are important for lipid transport. The chemical composition of each lipoprotein differs in that the HDL has a higher proportion of protein versus lipid, whereas the VLDL has a lower proportion of protein versus lipid.

As used herein, the term “triglyceride” means a lipid or neutral fat consisting of glycerol combined with three fatty acid molecules.

As used herein, the term “homocysteine” refers to an amino acid metabolite of S-adenosylmethionine degradation. Hyperhomocysteinemia is associated with chronic renal failure, hypothyroidism, breast, ovarian and pancreatic cancers, pernicious anemia and systemic lupus erythematosis. Elevated homocysteine levels are prevalent in CAD. It has been postulated that elevated homocysteine levels increase risk of CAD through direct toxicity to endothelial cells, increased coagulability, elevated triglyceride levels, and oxygen free radical production, which leads to higher endothelial reactivity with stimulation of smooth muscle cell proliferation.

As used herein, the term “C-reactive protein” refers to a serum protein whose function is not fully understood. It binds to a wide variety of substances, such as bacterial polysaccharide, phosphatidylcholine and damaged cell membranes. Several prospective studies have shown that C-reactive protein is a predictor of increased risk for myocardial infarction, stroke or peripheral vascular disease in asymptomatic individuals with no known coronary artery disease.

As used herein, the term “arginine compounds” means the naturally occurring L-amino acid, any physiologically acceptable salt, such as the hydrochloride salt, glutamate salt, etc., and any derivative thereof. Derivatives include peptides (i.e. poly L-arginine, arginine oligomers), other NO precursor such as homoarginine, or substituted arginine such as hydroxy-arginine. Naturally occurring sources include protamine.

As used herein, the term “policosanol” refers to mixtures of high molecular weight aliphatic alcohols ranging from about 20 to 40 carbons in length. Some major components of policosanol are octacosanol, triacontanol and dotriacontanol. Policosanol can be isolated from a number of different natural sources, including sugar cane wax, rice bran wax, and beeswax. As employed in the preferred embodiments, high molecular weight aliphatic alcohols of pharmaceutical grade can be obtained commercially and, preferably, pass extensive safety and efficacy procedures. One high molecular weight aliphatic alcohol product known as “Rice Bran Wax” is manufactured by Traco Labs, Inc. (Table 1).

TABLE 1
Composition of Rice Bran Aliphatic Alcohols from Traco Labs

| | Approximate Percentage of |
|---|---|
| Alipathic Alcohol | Total Aliphatic Alcohols |
| 1-C₂₂OH (1-Docosanol) | 1.3 |
| 1-C₂₄OH (1-Tetracosanol) | 11.5 |
| 1-C₂₆OH (1-Hexacosanol) | 10.5 |
| 1-C₂₈OH (1-Octacosanol) | 20.1 |
| 1-C₃₀OH (1-Triacontanol) | 30.0 |
| 1-C₃₂OH (1-Dotriacontanol) | 16.7 |
| 1-C₃₄OH (1-Tetratriacontanol) | 8.0 |
| 1-C₃₆OH (1-Hexatriacontanol) | 1.8 |

Another commercial source of high molecular weight aliphatic alcohols is Garuda International (Lemon Cove, Calif.). This company supplies several products comprising high molecular weight aliphatic alcohols. One of these products is sold under the brand name of LESSTANOL™ and comprises five aliphatic alcohols ranging from about 26 to 32 carbons in length (Table 2). An additional product from Garuda is OCTA-95, also isolated from sugar cane and comprises approximately 95 percent 1-octacosanol.

TABLE 2
Composition of Garuda Sugar Cane Wax Extract
LESSTANOL ™ Aliphatic Alcohols

| | Approximate Percentage of |
|---|---|
| Alipathic Alcohol | Total Aliphatic Alcohols |
| C₂₆OH (1-Hexacosanol) | 2 to 10 |
| C₂₇OH (1-Heptacosanol) | <0.5 |
| C₂₈OH (1-Octacosanol) | 55 to 65 |
| C₃₀OH (1-Triacontanol) | 15 to 30 |
| C₃₂OH (1-Dotriacontanol) | 2 to 10 |

As used herein, the term “high molecular weight aliphatic alcohols” means aliphatic alcohols having about 20 to 40 carbon atoms in length. High molecular weight aliphatic alcohols are components of policosanols. In the preferred embodiments, high molecular weight aliphatic alcohols are derived from plants, plant extracts, or other natural sources.

As used herein, “methyl donor cofactors” includes any natural or synthetic compound or any derivative thereof that functions as a cofactor with enzymes in intermediary metabolism to transfer or facilitate the transfer of a methyl group (—CH₃) and metabolic intermediates of such compounds. Examples of methyl donor cofactors include folic acid, trimethylglycine, vitamin B6 and vitamin B12 and their derivatives.

As used herein “folic acid derivatives” include naturally occurring or synthetic metabolic intermediates that may function as folate or substitute for folate in methyl transferase reactions. Examples of derivatives would include methylenetetrahydrofolate and 5-methyltetrahydrofolate.

As used herein “Vitamin B6” includes, respectively, pyridoxal, pyridoxine and pyridoxamine and naturally occurring or synthetic metabolic intermediates of these compounds as well as ethers or esters including phosphate or sulfate esters, and derivatives thereof.

As used herein “Vitamin B12” includes, respectively, cyanocobalamin, methylcobalamin, adenosylcobalamin and all naturally occurring or synthetic metabolic intermediates of these compounds and chemically altered variations of these compounds and derivatives thereof that can perform their metabolic functions relating to methyl group transfer.

“Conjugates” of arginine, aliphatic alcohols, and methyl donor cofactors means arginine forms, aliphatic alcohols, and methyl donor cofactors that are covalently bound or conjugated to a member selected from the group consisting of mono- or di-saccharides, amino acids, sulfates, succinate, and acetate. Preferably, the mono- or di-saccharide is a member selected from the group consisting of glucose, mannose, ribose, galactose, rhamnose, arabinose, maltose, and fructose.

The preferred embodiments provide a unique combination of arginine compounds, at lower doses than demonstrated in the prior art, with another component, such as high molecular weight aliphatic alcohols, to enhance and expand the serum lipid-lowering properties of arginine. The composition comprises, as a first component, at least one arginine compound or conjugate thereof, and as a second component, at least one member selected from the group consisting of high molecular weight aliphatic alcohol and methyl donor cofactors and derivatives thereof. Examples of suitable arginine compounds, high molecular weight aliphatic alcohols, and methyl donor cofactors are listed in Table 3, those containing at least one asterisk (*) are preferred and those containing two asterisks (**) are particularly preferred. The composition of the preferred embodiments can further comprise one or more members selected from the group consisting of antioxidants, vitamins, minerals, proteins, fats, carbohydrates or natural plant products. By attenuating multiple cardiovascular arterial disease risk factors, such as reducing serum total and LDL cholesterol, increasing serum HDL cholesterol, decreasing serum triglycerides, serum homocysteine and serum C-reactive protein, the present compositions and methods therefore significantly reduce the risks of CAD.

TABLE 3

| | High Molecular | |
|---|---|---|
| | Weight Aliphatic | Methyl Donor |
| Arginine Compounds | Alcohols | Cofactors |
| Arginine** | 1-C₂₀OH | Folic Acid** |
| Homoarginine** | 1-C₂₂OH* | Trimethylglycine* |
| Magnesium arginate** | 1-C₂₄OH* | Vitamin B6** |
| Chromium arginate* | 1-C₂₆OH* | Vitamin B12** |
| Arginine oligomers | 1-C₂₈OH** | 5-Methyl Tetrahydrofolate |
| Protamine | 1-C₃₀OH** | Methylene Tetrahydrofolate |
| L-hydroxy-arginine | 1-C₃₂OH** | |
| acetate** | | |
| Arginine aspartate | 1-C₃₄OH* | |
| | 1-C₃₆OH* | |
| | 1-C₃₈OH | |
| | 1-C₄₀OH | |

A certain embodiment is a composition comprising at least one arginine compound or conjugate thereof and at least one member selected from the group consisting of 1-octacosanol, 1-triacontanol, 1-dotriacontanol, folic acid, vitamin B6, vitamin B12, trimethylglycine, 5-methyl tetrahydrofolate and methylene tetrahydrofolate.

In the preferred embodiments, the ratio of arginine compounds to high molecular weight aliphatic alcohols is preferably from about 1:1 to about 1,000:1. The ratio of arginine compounds to folate or derivatives is preferably from about 1:10 to about 20,000:1. The ratio of arginine compounds to vitamin B6 or derivatives is preferably from about 1:2 to about 200:1. The ratio of arginine compounds to vitamin B12 or derivatives is preferably from about 1:2 to about 416,667:1. The ratio of arginine compounds to trimethylglycine is preferably from about 1:1 to about 5000:1.

The composition of the preferred embodiments is formulated to deliver about 0.5 to 2,000 mg of an arginine compound or conjugate thereof and another component selected from about 0.5 to 100 mg of high molecular weight aliphatic alcohol or about 0.0001 to 10000 mg of a methyl donor cofactor. Preferably, the composition of the preferred embodiments is formulated to deliver about 1 to 1,000 mg of an arginine compound or conjugate thereof and another component selected from about 1 to 50 mg of high molecular weight aliphatic alcohol or about 0.01 to 5000 mg of a methyl donor cofactor.

Preferably, the composition of the preferred embodiments is formulated to deliver about 0.5 to 2,000 mg of an arginine compound or conjugate thereof and another member selected from about 0.5 to 100 mg of high molecular weight aliphatic alcohol; about 0.01 to 50 mg folic acid; about 1 to 6000 mg vitamin B6, about 0.0001 to 5 mg vitamin B12; and about 0.0001 to 5 mg trimethylglycine. In a certain embodiment, the composition is formulated to deliver about 1 to 1,000 mg of an arginine compound or conjugate thereof and another member selected from about 1 to 50 mg of high molecular weight aliphatic alcohol; about 0.05 to 10 mg folic acid; about 5 to 3000 mg vitamin B6, about 0.0024 to 2 mg vitamin B12; and about 0.0024 to 2 mg trimethylglycine.

Arginine is a nonessential amino acid that can be found in soy proteins. Setting arginine apart from other amino acids was the discovery that it serves as the substrate for nitric oxide synthetase (NOS). Arginine plus molecular oxygen are converted by NOS to NO plus citrulline. FADH, FAD and FMNH2 are cofactors for the reaction. NO has been identified as a critical signaling molecule in maintaining blood pressure in the cardiovascular system, in modulating neural transmission in the brain, and in stimulating host defenses in the immune system. Recent evidence further suggests that NO may be involved in regulation of gene expression, learning and memory, platelet aggregation, male sexual function, cytotoxicity and in stimulating apoptosis. Furthermore, reduced NO levels have been associated with development of atherosclerosis. Abundant research reports have demonstrated the ability of intravenous or oral arginine to lower serum lipid levels. The use of arginine for prophylaxis and therapy in atherosclerosis is taught in U.S. Pat. No. 5,428,070. While apparently effective in increasing NO production, however, suggested quantities of arginine are untenable dose regimens and are costly. Combination of arginine with other compounds of the preferred embodiments that can reduce the dose of arginine necessary to achieve prophylaxis and therapy in the atherosclerotic process would be novel and appreciated.

Use of high molecular weight aliphatic alcohols for treatment of hypercholesterolemia has been disclosed in U.S. Pat. Nos. 5,856,316 and 5,663,156, which describe a process of preparation from sugar cane and a mixture of higher primary aliphatic alcohols from about 24 to 34 carbons having a particular quantitative combination. Sorkin (U.S. Pat. Nos. 5,952,393 and 6,197,832) discloses a composition comprising phytosterol and policosanol and methods of use thereof for reducing serum cholesterol in humans and animals. Perez (U.S. Pat. No. 6,225,354) describes a mixture of higher molecular weight aliphatic alcohols naturally obtained from beeswax that contain about 24 to 34 carbon atoms. Mixtures of aliphatic alcohols of about 20 to 40 carbons in length are found in natural sources and have demonstrated the ability to lower serum total and LDL cholesterol. One particular combination of these fatty alcohols has been termed policosanol, a mixture of high molecular weight aliphatic alcohols, generally ranging from about 24 to 34 carbons in length. These long-chain alcohols can be extracted from rice bran, sugar cane wax, or beeswax. The profile of the aliphatic alcohols differs somewhat depending upon source and method of extraction. However, it is believed that the serum cholesterol-lowering effect is attributable primarily to octacosanol, triacontanol, and dotriacontanol content of the extract.

High molecular weight aliphatic alcohols function by inhibiting the synthesis of cholesterol in the liver and increasing the hepatic reabsorption of LDL (bad cholesterol). Double-blind control studies, involving a total of almost 1500 individuals and ranging in length from 6 weeks to 12 months, have found high molecular weight aliphatic alcohols effective for improving cholesterol levels. The results suggest that treatment with as little as about 10 mg high molecular weight aliphatic alcohols per day can reduce LDL cholesterol by about 20 percent or more and total cholesterol by about 15 percent. Some studies found improvement in triglyceride and HDL (good) cholesterol, but others did not Interestingly, most of these studies enrolled only individuals whose cholesterol levels had not improved with diet alone. A formulation of high molecular weight aliphatic alcohols that consistently lowers serum triglyceride levels is needed to establish these aliphatic alcohols as beneficial for reducing multiple risk factors in CAD.

Typical clinical doses of high molecular weight aliphatic alcohols used to lower the elevated serum cholesterol range from about 5 to 10 mg administered twice daily. Several weeks, e.g. two months, of treatment may be required for noticeable results to develop. High molecular weight aliphatic alcohols appear to be safe at recommended doses. In the published clinical studies, only mild, short-term side effects such as nervousness, headache, diarrhea, and insomnia were seen. High molecular weight aliphatic alcohols appear to enhance the blood-thinning effects of aspirin, suggesting that unsupervised combination therapy could be dangerous. By the same principle, high molecular weight aliphatic alcohols should not be combined with other blood-thinning drugs, such as warfarin, heparin or pentoxifylline. There is also a chance that they might cause excessive bleeding if combined inappropriately with natural supplements that reduce clotting time, such as garlic, ginkgo and high doses of vitamin E. Because of the potential risks of adverse interactions with other medications and products used for the modification of cardiovascular risk factors, there is a need for a formulation containing long-chain fatty alcohols that adequately address multiple cardiovascular risk factors safely and effectively.

The group of vitamins that are methyl group donor cofactors include folic acid, vitamin B6 and vitamin B12. These enzyme cofactors function in methyl group donor reactions critical to DNA and protein regulation. Without adequate amounts of the methyl donor cofactors such as folate, vitamins B6 and B12, homocysteine accumulates. Increased levels of homocysteine have been associated with coronary heart disease. Vitamin B6 allows for the formation of gluthathione from homocysteine, while vitamin B12 and folate recycle homocysteine to methionine to allow the cycle to S-adenosylmethionine to continue.

For most uses, folate dosing is about 400 μg/day with a range of about 50 to 10,000 μg/day. Vitamin B6 doses commonly range from about 5 to 300 mg per day, higher than the basic nutritional requirement of about 1 to 2 mg per day. Vitamin B12 is extremely safe and has been given to the elderly in doses ranging from 2.4 μg/day to 2000 μg/day as a dietary supplement (lower end of range) or to correct B12 deficiencies (higher end of range).

In addition to the active ingredients described, the present dietary supplement can include various additives such as other natural components of intermediary metabolism, vitamins, minerals and natural plant products. Examples of natural plant products would include tea, coffee, ephedrine, and phytosterols. Such additions would function to enhance the cardiovascular risk modification capacity of the formulation. Other inert ingredients such as magnesium stearate that are standard excipients in the manufacture of tablets and capsules would also be obvious to those skilled in the art of pharmaceutical manufacturing.

As used herein, “pharmaceutically acceptable carrier” includes any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrates, coloring agents, bulking agents, flavoring agents, sweetening agents and miscellaneous materials such as buffers and absorbents that may be needed in order to prepare a particular therapeutic composition. The use of such media and agents for pharmaceutically active substances is well known in the art. Except insofar as any conventional media or agent is incompatible with the active ingredients, its use in the present composition is contemplated. In one embodiment, talc and magnesium stearate, are included in the present formulation. Other ingredients used to affect the manufacture of this composition, as a dietary bar or functional food, can include flavorings, sugars, aminosugars, proteins and/or modified starches, as well as limited fats and oils.

The dietary supplement or therapeutic composition of the preferred embodiments can be formulated in any manner known by one of skill in the art. In one embodiment, the composition is formulated into a capsule, caplet, tablet, or softgel using techniques available to one of skill in the art. However, provided the proper daily dosage is incorporated, the present compositions may also be formulated in other convenient forms, such as a solution or suspension, a spray solution or suspension, a liquid, a food, or snack item. Food, snack, or liquid items can include any ingestible ingredients, including sweeteners, flavorings, oils, starches, proteins, fruits or fruit extracts, vegetables or vegetable extracts, grains, animal fats or proteins. Thus, the present compositions can be formulated into cereals, snack items such as chips, bars, gumdrops, or chewable candies.

The preferred embodiments also provide methods for reduction of multiple CAD risk factors in a warm-blooded animal. The method comprises administering to the animal the compositions of the preferred embodiments for a period of time and in an amount sufficient to reduce serum levels of total cholesterol and LDL cholesterol, increase serum levels of HDL cholesterol, and reduce serum levels of triglycerides, homocysteine and C-reactive protein.

Since many modifications, variations and changes in detail can be made to the described preferred embodiments, it is intended that all matters in the foregoing description and the following examples are interpreted to illustrate and not in any way to be limiting.

### EXAMPLE 1

### A Tablet Formulation Containing L-Arginine, High Molecular Weight Aliphatic Alcohols, and Methyl Donor Cofactors

The formulation listed in TABLE 4 would be administered once or twice per day and be expected to efficiently address the CAD risk factors of serum cholesterol (total, LDL and HDL), triglycerides, homocysteine and C-reactive protein.

TABLE 4

| | | Amount |
|---|---|---|
| | Amount | [Approximate % of |
| Component | [mg] | Actives] |
| High molecular weight aliphatic alcohols | 5 | 1.0 |
| L-Arginine | 500 | 96.5 |
| Folate | 0.400 | 0.1 |
| Vitamin B6 | 12.5 | 2.4 |
| Vitamin B12 | 0.500 | 0.1 |
| Calcium phosphate (base) | 11 | — |
| Cellulose (tablet coating) | 2 | — |
| Stearic acid (lubricant) | 1 | — |
| Magnesium stearate (lubricant) | 0.3 | — |
| Silicon dioxide (diluent) | 0.4 | — |

### EXAMPLE 2

### A Formulation Containing Arginine and High Molecular Weight Aliphatic Alcohols

The formulation listed in TABLE 5 would be administered once or twice per day as a tablet and be expected to efficiently address the CAD risk factors of serum cholesterol (total, LDL and HDL), triglycerides, and C-reactive protein.

TABLE 5

| | | Amount |
|---|---|---|
| | Amount | [Approximate % of |
| Component | [mg] | Actives] |
| High molecular weight aliphatic alcohols | 5 | 1.1 |
| Arginine | 500 | 98.9 |
| Calcium phosphate (base) | 5 | — |
| Cellulose (tablet coating) | 1 | — |
| Stearic acid (lubricant) | 0.500 | — |
| Magnesium stearate (lubricant) | 0.150 | — |
| Silicon dioxide (diluent) | 0.200 | — |

### A Formulation Containing Homoarginine and Methyl Donor Cofactors

The formulation listed in TABLE 6 would be administered once or twice per day as a tablet and be expected to efficiently address the CAD risk factors of serum cholesterol (total, LDL and HDL), homocysteine, and C-reactive protein.

TABLE 6

| | | Amount |
|---|---|---|
| | Amount | [Approximate |
| Component | [mg] | % of Actives] |
| Homoarginine | 500 | 90.6 |
| Trimethylglycine | 1.00 | 0.2 |
| Vitamin B6 | 50 | 9.1 |
| Vitamin B12 | 1.00 | 0.2 |
| Calcium phosphate (base) | 11 | — |
| Cellulose (tablet coating) | 2 | — |
| Stearic acid (lubricant) | 1 | — |
| Magnesium stearate (lubricant) | 0.3 | — |
| Silicon dioxide (diluent) | 0.4 | — |

### EXAMPLE 4

### Attenuation of Risk Profile in Patients with Multiple Cardiovascular Risk Factors by Two Formulations of High Molecular Weight Alcohols

One-hundred patients of both sexes, aged 40 to 80 years, with a history of multiple cardiovascular risk factors are enrolled in the study after providing informed written consent. The study protocol is approved by an independent institutional review board. Any lipid-lowering therapy or drug with a recognized effect on cardiovascular risk factors is discontinued from the time the patient is recruited until the completion of the study. Exclusion criteria include active renal disease, diagnosed neoplastic diseases, severe hypertension (diastolic pressure>120 mmHg) or uncontrolled diabetes. Additionally, patients who have suffered myocardial infarction, stroke or coronary surgery within three months of the study are also excluded.

At the start of the trial, patients are given a physical examination and randomized to receive the formulation described in TABLE 4 (Test) or a tablet containing 5 mg of the policosanols profile described by Granja (U.S. Pat. No. 5,853,316 and U.S. Pat. No. 5,663,156; GF1). The source of policosanols used in the TABLE 4 formulation is Garuda INTERNATIONAL. Profiles of the aliphatic alcohol composition in GF1 and Test are presented in TABLE 7. Formulation GF1 contains only the high molecular weight aliphatic alcohols as active ingredients. The trial is double-blinded in that neither the patient nor the physician knows the identity of the formulation being used. Patients are instructed to take the tablet (GF1 or Test) twice daily with the evening meal for eight weeks. After the eight-week study period, laboratory tests are repeated, a second physical examination is performed, compliance assessed, and adverse effects noted.

TABLE 7
Formulations of Aliphatic Alcohols Used in Trial

| Granja Formulation 1 | | Test Formulation | |
|---|---|---|---|
| | Approximate | | Approximate |
| Alcohol | Percent | Alcohol | Percent |
| 1-tetracosanol | 0.8 | 1-tetracosanol | — |
| 1-hexacosanol | 6.7 | 1-hexacosanol | 5.1 |
| 1-heptacosanol | 3.0 | 1-heptacosanol | <0.5 |
| 1-octacosanol | 65.6 | 1-octacosanol | 62.0 |
| 1-nonacosanol | 0.7 | 1-nonacosanol | — |
| 1-triacontanol | 12.5 | 1-triacontanol | 18.0 |
| 1-dotriacontanol | 5.0 | 1-dotriacontanol | 8.0 |
| 1-tetratriacontanol | 0.8 | 1-tetratriacontanol | — |

All blood samples are taken between 8:00 and 8:30 a.m. after a 12-hour fast. Blood biochemistry for lipid profile analysis, homocysteine and C-reactive protein are determined by colorimetric enzymatic methods using commercially available reagent kits. Laboratory safety tests include determination of glucose, creatinine, aspartate aminotransferase, and alanine aminotransferase, and are performed using routine colorimetric enzymatic methods provided in commercially available reagent kits.

Within-group comparisons of continuous variables are performed using the Wilcoxon test for paired samples; between group comparisons are performed using the Nann Whitney U-test. All tests are two-tailed and performed using the CSS statistical package (Stat Soft, Tulsa, Okla.).

No significant differences exist between GF1 and Test groups at randomization. Five patients left the study for reasons unrelated to the test materials, three from the GF1 and two from the Test group. TABLE 8 lists the changes in CAD risk factors over the eight-week study period for both groups. Differences in total cholesterol, LDL cholesterol and HDL cholesterol are similar for both formulations, with somewhat larger changes occurring with Test. Dramatic differences between formulations are seen, however, with respect to depression of serum triglycerides, homocysteine and C-reactive protein. Test is able to favorably modify these CAD risk factors, while GF1 is not.

TABLE 8
Approximate Percent Change In Serum Cardiovascular Risk Factors
Over The Eight-Week Period For The Gf1 And Test Formulations

| | |
|---|---|
| Cardiovascular | Formulation |

| Risk Factor | GF1† [n = 47] | Test† [n = 48] |
|---|---|---|
| Total cholesterol | −12.6 | −15.2 |
| LDL-cholesterol | −14.1 | −21.7** |
| Triglycerides | −0.2 | −10.1* |
| HDL-cholesterol | +16.7 | +22.0 |
| Homocysteine | +4.5 | −22.4** |
| C-reactive protein | −5.6 | −15.4** |

†Values are the percent change from pre-study levels.
*/**Significantly different from GF1 with p < 0.05 and <0.01, respectively.

Many modifications and variations of the embodiments described herein may be made without departing from the scope, as is apparent to those skilled in the art. The specific embodiments described herein are offered by way of example only.

1. A composition for coordinately attenuating multiple cardiovascular arterial disease risk factors comprising at least one arginine compound or conjugate thereof and at least one member selected from the group consisting of high molecular weight aliphatic alcohol and methyl donor cofactor and conjugates thereof.

2. The composition according to claim 1, wherein the arginine compound is a compound selected from the group consisting of arginine, homoarginine, magnesium arginine, chromium arginine, arginine oligomers, protamine, L-hydroxy-arginine acetate, and arginine aspartate.

3. The composition according to claim 1, wherein the high molecular weight alcohol is an alcohol selected from the group consisting of 1-C₂₀OH, 1-C₂₂OH, 1-C₂₄OH, 1-C₂₆OH, 1-C₂₈OH, 1-C₃₀OH, 1-C₃₂OH, 1-C₃₄OH, 1-C₃₆OH, 1-C₃₈OH, and 1-C₄₀OH.

4. The composition according to claim 1, wherein a ratio of arginine compound to high molecular weight alcohol is from about 1:1 to 1,000:1.

5. The composition according to claim 1, wherein the methyl donor cofactor is a compound selected from the group consisting of folic acid, trimethylglycine, vitamin B6, vitamin B12, 5-methyl tetrahydrofolate, methylene tetrahydrofolate, and derivatives thereof.

6. The composition according to claim 5, wherein a ratio of arginine compound to folic acid or derivatives is from about 1:10 to 20,000:1.

7. The composition according to claim 5, wherein a ratio of arginine compound to vitamin B6 or derivatives is from about 1:2 to 200:1.

8. The composition according to claim 5, wherein a ratio of arginine compound to vitamin B12 or derivatives is from about 1:2 to 416,667:1.

9. The composition according to claim 5, wherein a ratio of arginine compound to trimethylglycine or derivatives is from about 1:1 to 5000:1.

10. The composition according to claim 1, wherein the composition comprises the arginine compound from about 0.5 to 2000 mg.

11. The composition according to claim 1, wherein the composition comprises the high molecular aliphatic alcohol from about 0.5 to 100 mg.

12. The composition according to claim 1, wherein the composition comprises the methyl donor cofactor from about 0.0001 to 10000 mg.

13. The composition according to claim 1, further comprising a member selected from the group consisting of antioxidants, vitamins, minerals, proteins, fats, carbohydrates, and natural plant products.

14. The composition according to claim 1, wherein the composition is in a form selected from the group consisting of solid capsules, caplets, tablets, softgels, liquids, bars, and functional foods.

15. A composition for coordinately attenuating multiple cardiovascular arterial disease risk factors comprising at least one arginine compound or conjugate thereof and at least one compound selected from the group consisting of 1-octacosanol, 1-triacontanol, 1-dotriacontanol, folic acid, vitamin B6, vitamin B12, trimethylglycine, 5-methyl tetrahydrofolate and methylene tetrahydrofolate.

16. The composition according to claim 15, wherein the arginine compound is a compound selected from the group consisting of arginine, homoarginine, magnesium arginine, chromium arginine, arginine oligomers, protamine, L-hydroxy-arginine acetate, and arginine aspartate.

17. A method for dietary supplementation to a warm blooded animal comprising administering to the animal an effective amount of a composition comprising at least one arginine compound or conjugate thereof and at least one member selected from the group consisting of high molecular weight aliphatic alcohol and methyl donor cofactor and conjugates thereof.

18. The method according to claim 17, wherein the dietary supplementation is continued until a serum level of a cardiovascular arterial disease risk factor selected from the group consisting of cholesterol, triglyceride, homocysteine, and C-reactive protein is decreased.

19. The method according to claim 17, wherein the dietary supplementation is continued until a serum level of HDL-cholesterol is increased.

20. The method according to claim 17, wherein the dietary supplementation is used to treat a medical condition selected from the group consisting of hyperchoesterolemia, hypertryglyceridemia, hyperhomocysteinemia, and elevated levels of C-reactive protein.

21. The method according to claim 17, wherein the dietary supplementation is used to prevent a medical condition selected from the group consisting of hyperchoesterolemia, hypertriglyceridemia, hyperhomocysteinemia, and elevated levels of C-reactive protein.

22. The method according to claim 17, wherein the arginine compound is a compound selected from the group consisting of arginine, homoarginine, magnesium arginine, chromium arginine, arginine oligomers, protamine, L-hydroxy-arginine acetate, and arginine aspartate.

23. The method according to claim 17, wherein the high molecular weight alcohol is an alcohol selected from the group consisting of 1-C₂₀OH, 1-C₂₂OH, 1-C₂₄OH, 1-C₂₆OH, 1-C₂₈OH, 1-C₃₀OH, 1-C₃₂OH, 1-C₃₄OH, 1-C₃₆OH, 1-C₃₈OH, and 1-C₄₀OH.

24. The method according to claim 17, wherein the methyl donor cofactor is a compound selected from the group consisting of folic acid, trimethylglycine, vitamin B6, vitamin B12, 5-methyl tetrahydrofolate, methylene tetrahydrofolate, and derivatives thereof.

25. The method according to claim 17, further comprising a member selected from the group consisting of antioxidants, vitamins, minerals, proteins, fats, carbohydrates, and natural plant products.

26. The method according to claim 17, wherein the composition is in a form selected from the group consisting of solid capsules, caplets, tablets, softgels, liquids, bars, and functional foods.
